(19) United States
(12) Patent Application Publication
(10) Pub. No.: US 2010/0241170 A1
(43) Pub. Date: Sep. 23, 2010

(54) ANCHOR ASSEMBLY FOR SPINAL IMPLANT SYSTEM

(76) Inventors: Frank Cammisa, Greenwich, CT (US); Javin Pierce, Stowe, VT (US)

Correspondence Address: DARBY & DARBY P.C., P.O. BOX 770, Church Street Station, New York, NY, 10008-0770, US

(21) Appl. No.: 12/444,124

(22) PCT Filed: Oct. 3, 2007
(86) PCT No.: PCT/US2007/080283

§ 371 Date: Jun. 4, 2010

Related U.S. Application Data
(60) Provisional application No. 60/850,373, filed on Oct. 5, 2006.

(51) Int. Cl. A61B 17/70; A61B 17/86

(52) U.S. Cl. 606/264; 606/305; 606/302

(57) ABSTRACT

An anchor assembly for use with a fixation system for stabilizing one or more spinal bone segments by means of an elongated stabilizer includes an anchor base that receives a fastener and includes s a first coupling feature including guide slots and a recessed channel for receiving the elongated stabilizer. The anchor assembly further includes a cap that has a second coupling feature that is complementary to the first coupling feature and includes structures that are laterally received in the base guide slots for mating the cap to the base, as well as a stop element that in combination with the first coupling feature limits the lateral movement of the cap in the guide slots. The cap includes a retaining element that is driven into engagement with the elongated stabilizer so as to lock and retain the elongated stabilizer in place within the anchor assembly.

### CROSS-REFERENCE TO RELATED APPLICATION

This application claims the benefit of U.S. patent application Ser. No. 60/850,373, filed Oct. 5, 2006, which is hereby incorporated by reference in its entirety.

### TECHNICAL FIELD

The present invention relates generally to an apparatus for immobilizing a spine and more particular to a fixation, anchor or implant device that can be used in an apparatus for posterior internal fixation of the spine.

### BACKGROUND

Over the years, a number of different methods of spinal immobilization have been developed and used for the treatment of spinal instability and displacement. For example, one method involves the immobilization of the joint by surgical fusion or the like and yet another class of treatment is post operative external immobilization, such as the use of splints and casts. However, as the complexity of the surgical procedures grew, other methods of internal and external fixation were developed in an attempt to provide a more effective method for stabilizing the spine.

Internal fixation refers to therapeutic methods of stabilization which are wholly internal to the patient and typically include the use of bone plates and pins to immobilize the spine, while external fixation involves at least some portion of the stabilizing device being external to the body of the patient. Today, internal fixation is the preferred method for immobilizing the spine since it permits the patient to have a greater degree of movement and comfort since the components of the device are internal to the patient.

One of the greatest challenges in implementing the internal immobilization method is the matter of how to secure the fixation device to the spine without damaging the spinal cord itself. Traditionally, the pedicles are a favored area for attaching the device since the pedicles offer an area that is strong enough to hold the fixation device even in the case where the patient suffers from osteoporosis. In earlier designs, screws were used to attach the device, with the screws extending through the facets into the pedicles. More recently, posterior methods have been developed where wires extend through the spinal canal and hold a rod against the lamina or that utilize pedicular screws which extend into the pedicle and secure a plate which extends across several vertebral segments.

U.S. Pat. No. 5,474,555, which is hereby incorporated by reference in its entirety, discloses an apparatus for the internal fixation of the spine that consists of a rod and vertebral anchors which hold the rod in place at a chosen location. Each anchor is secured to the vertebrae by a transpedicular screw member. More specifically, the apparatus of the '555 utilizes two implant sets on either side of the spinous processes. Each implant set includes a stainless steel rod that spans the vertebrae to be immobilized. One implant set is used on one side of the spinous process on the posterior side of the lamina and the transverse process. The rod is held in place in position by a stainless steel vertebral anchor which captures the rods. The anchor has a set member which is secured to the vertebrae by a stainless steel transpedicular screw. The screw is separate from the anchor seat and thus provides for limited motion between the anchor seat and the vertebrae.

While, the apparatus of the '555 is satisfactory for its intended use, the apparatus includes a number of different, separate parts that have to be connected together to secure the apparatus to the spine. In addition, by having a number of different parts, the '555 apparatus requires a number of different tools to be used in order to fix the apparatus in its intended place.

Thus, there is a need and a desire to provide an apparatus that offers an alternative design to the '555 design and overcomes some of the shortcomings of the '555 design by providing a simpler, less complex design that also requires less time in fixing the apparatus to the bony surface and in locking the elongated stabilizer. Furthermore, there is an unmet need for more compact implants that can be reliably applied by means of less invasive surgical techniques to minimize the morbidity and rehabilitation time associated with the procedure. Additionally, there is a major benefit to providing an implant that is more compact to improve long-term patient comfort and to reduce tissue impingement.

### SUMMARY

According to one aspect of the present invention, an anchor assembly for use with a fixation system for stabilizing one or more spinal bone segments by means of an elongated stabilizer includes a fastener for securing the anchor assembly to the spinal bone segment and an anchor base that includes a first cavity and a first bore with a seat for receiving a head of the fastener such that the fastener extends through first bore and extends outwardly therefrom. The anchor base has a first coupling feature including guide slots and a recessed channel for receiving the elongated stabilizer.

The anchor assembly further includes a cap that has a threaded bore formed therethrough. The cap has a second coupling feature including rail structures that permit the cap to be laterally received in the guide slots, as well as a stop element that in combination with the first coupling feature limits the lateral movement of the cap in the guide slots in a first position and causes the first bore and the threaded bore to be aligned in the first position. The anchor assembly further includes a retaining element that is threadingly received within the threaded bore of the cap and can be driven into engagement with the elongated stabilizer so as to lock and retain the elongated stabilizer in place within the anchor assembly.

According to another embodiment, an anchor assembly for use with a fixation system for stabilizing one or more spinal bone segments by means of an elongated stabilizer includes a fastener for securing the anchor assembly to the spinal bone segment and an anchor base that includes a first cavity and a first bore with a seat for receiving a head of the fastener such that the fastener extends through first bore and extends outwardly therefrom. The anchor base has a first coupling feature including guide slots and shaped slots for receiving the elongated stabilizer.

A cap assembly is constructed to mate with the anchor base and includes a base having a threaded post and a second coupling feature including structures that permit the cap to be laterally received and moved within the guide slots while vertical movement of the cap is restricted. The cap assembly includes a cap fastener that is threadingly mated with the threaded post and can be driven along the threaded post into engagement with the elongated stabilizer so as to lock and retain the elongated stabilizer in place within the anchor assembly.

Further aspects and features of the exemplary automated safety cap removal mechanism disclosed herein can be appreciated from the appended Figures and accompanying written description.

### BRIEF DESCRIPTION OF THE DRAWING FIGURES

FIG. 1 is a side view of a spine with an anchor assembly according to the present invention in place;

FIG. 2 is a perspective view of an anchor assembly according to a first embodiment of the present invention;

FIG. 3 is a first exploded perspective view of selected components of the assembly of FIG. 2;

FIG. 4 is a second exploded perspective view of the assembly of FIG. 2;

FIG. 5 is a third exploded perspective view of the assembly of FIG. 2;

FIG. 6 is a side perspective view of the assembly of FIG. 2;

FIG. 7 is a perspective view of an anchor assembly according to a second embodiment of the present invention;

FIG. 8 is a cross-sectional view of the assembly of FIG. 7;

FIG. 9 is an exploded perspective view of the assembly of FIG. 7;

FIG. 10 is a side exploded perspective view of the assembly of FIG. 7;

FIG. 11 is a side perspective view of a cap base of the assembly of FIG. 7;

FIG. 12 is another side perspective view of the cap base;

FIG. 13 is a side view of the cap base; and

FIG. 14 is a side perspective view of an anchor base of the assemblies of FIGS. 1 and 7.

### DETAILED DESCRIPTION OF PREFERRED EMBODIMENTS

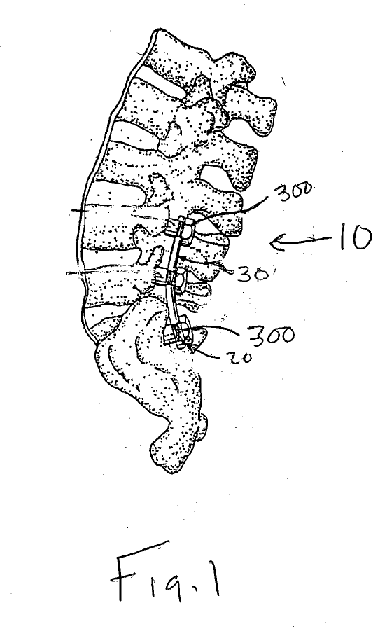

FIG. 1 is a side view of a spine with an anchor assembly according to the present invention in place and described in greater detail below.

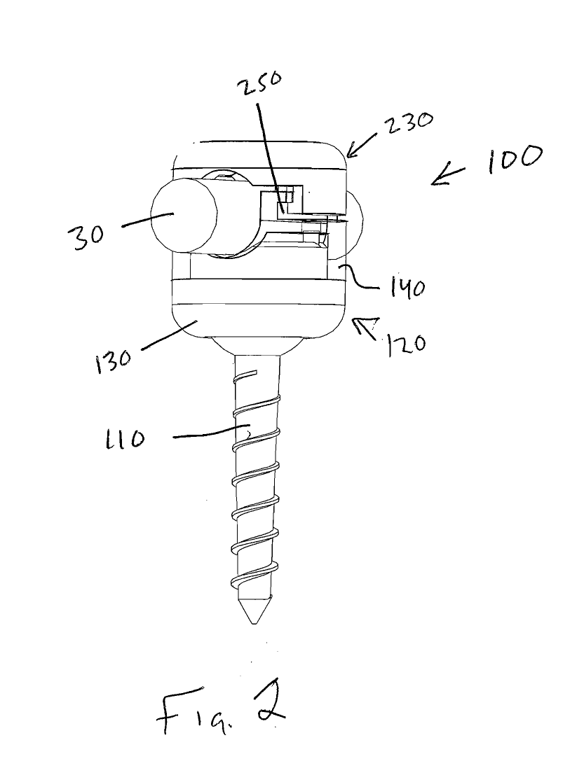

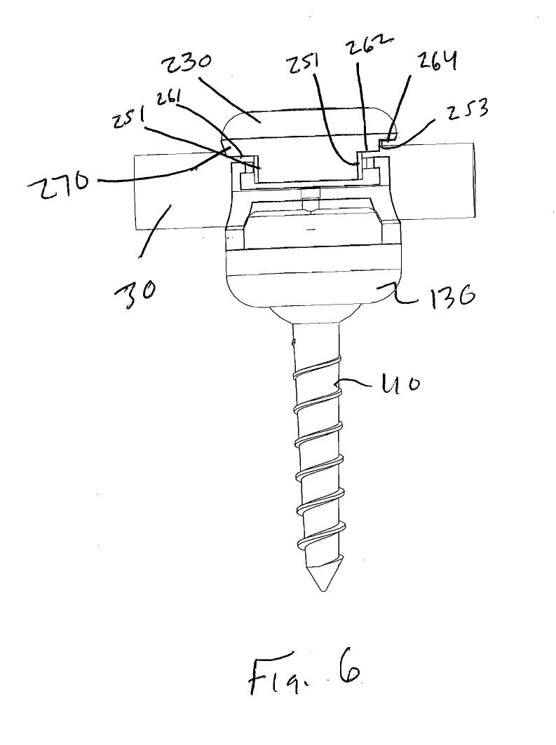

FIGS. 2-6 and 14 illustrate a fixation device or anchor assembly 100 according to one embodiment for use in a system 10 for the internal fixation of the spine. The system 10 is generally in the form of an anchor screw and rod system as disclosed in U.S. Pat. No. 5,474,555, which has been previously incorporated by reference in its entirety. However, it will be understood that the anchor assembly 100 according to the present invention can be used with other types of apparatus and systems that are designed to immobilize the spine, including the lumbosacral region.

The system 10 utilizes two implant sets 20 that are positioned on either side of the spinous process. Each set 20 includes a plurality of anchor assemblies 100, 300 and a rod 30 that is of sufficient length to span the length of the spine to be immobilized. One anchor assembly 100, 300 is positioned on the dorsal side of the vertebra and in general, a separate anchor assembly 100, 300 is used for each vertebra (representing the length of spine to be stabilized). The rod 30 is held by the anchor assemblies 100, 300 posterior to the vertebra. The rod 30 is formed of a suitable biocompatible material having a sufficient strength, such as quarter inch stainless steel rod. The rod 30 should be able to withstand lateral bending forces and torsion since the system may be used to correct spinal displacement and curvature. However, the rod 30 should be capable of being bent intraoperatively to a certain extend so that the rod can assume the proper curvature for the individual application.

The anchor assembly 100 according to one exemplary embodiment of the present invention is illustrated in FIGS. 2-6 and 14. The anchor 100 is actually an assembly of different parts including a pedicle fastener 110, such as a transpedicular screw, that is intended to anchor or fix the anchor assembly 100 into the pedicle of a patient or some other target location. The pedicle fastener 110 can be formed of any suitably strong biocompatible material, such as stainless steel, and the fastener 110 can be a standard stainless steel cancellous screw with a thread of a given diameter. The fastener 110 can be provided in any number of different lengths so long as it is intended to be accommodated by average adult pedicles of the lower thoracic, lumbar and the upper two sacral vertebral segments.

The fastener 110 includes a head 112 and a shank 114 that is above a threaded section 116 and joins a rounded shoulder 118 of the head 112. The head 112 of the fastener 110 is constructed so that it can be driven with a tool, such as a hex driver, into the target location in the body, such as the pedicle.

The anchor assembly 100 also includes a base 120 that receives the fastener 110 and is securely attached to the pedicle by means of driving the fastener 110 into the pedicle. It will be appreciated that the base 120 can be formed as a single part or it can be formed of two parts that are joined together as shown in FIG. 2-6. More specifically, the illustrated base 120 includes a first part 130 (lower part) and a second part 140 (lower part) that are joined together using any number of techniques, including a mechanical fit (e.g., snap-fit, frictional fit, etc.) or by any number of different bonding techniques, such as a weld, etc. The first and second parts 130, 140 are both generally circular shaped structures.

The first part 130 includes a central bore 132 that extends therethrough and is sized to receive the elongated threaded section 116. An annular shaped beveled section 134 defines the bore 132 and is inwardly tapered toward a bottom face 131 of the first part 130. The beveled section 134 defines a seat for engaging the rounded shoulder 118 of the head 112; however, the diameter of the central bore 132 is less than the diameter of the head 112 and therefore, the head 112 has a limited degree of travel within the first part 120 and is prevented from passing completely through the central bore 132. The radius of curvature of the beveled section 134 is thus complementary to the radius of curvature of the rounded shoulder 118 of the fastener head 112 so that when the fastener 110 is received through the bore 132, the head 112 seats flush against the beveled section 134 that acts as a stop.

Above the beveled section 134, the first part 130 includes an annular planar platform or floor 150 that is defined in part by an outer peripheral wall 152 that extends vertically therefrom, preferably at a right angle. An upper edge of the wall 152 defines a top face of the first part 130. The top face of the fastener head 112 can be below or above the plane that is defined by and contains the top face of the first part 130.

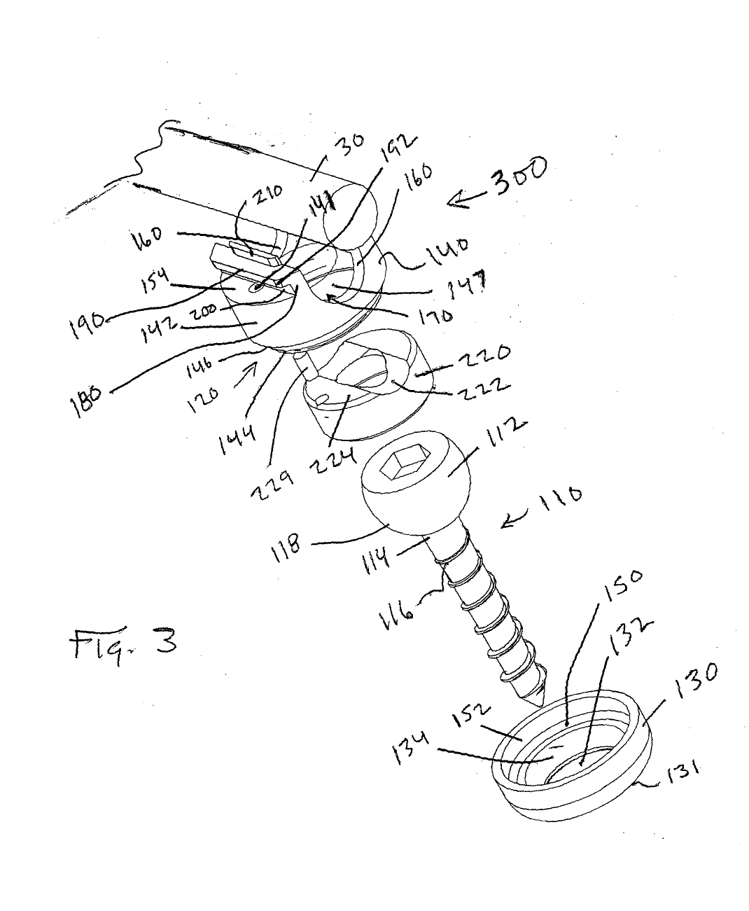

As best shown in FIGS. 3 and 14, the second part 140 includes a hollow body 142 that has a lower annular flange 144 that is defined by a vertical wall 145 that extends up to a shoulder 146 (e.g., right angle shoulder). Similar to the first part 130, the second part 140 has a central bore 147 that extends therethrough. The right angled shoulder 146 is designed so that when the first and second parts 130, 140 mate together, the top edge of the wall 152 seats within the shoulder 146 so that the outer surfaces of the first and second parts 130, 140 are flush with one another.

The width of the annular section 144 is less than or about equal to the width of the annular floor 150 since when the second part 140 mates with the first part 130 by being received therein, the annular section 144 seats on the floor 150.

The hollow body 142 has a pair of arcuate surfaces 160 (e.g., semi-circular shaped) that are formed opposite one another to form a U-shaped channel 170 that extends across the central bore 147 of the hollow body 142 and is constructed so that the rod 30 can be received and securely held within the channel 170 above a cap 310 and below by a shim 220. In other words, the width of the rod 30 is always slightly less than the width of the channel 170. Furthermore, the channel 170 is disposed far enough below an arcuate channel 222 of the shim 220 so that the rod 30 will not contact the bottom of the channel 170 during or after final fixation of the system.

The hollow body 142 has an upper planar face 154 that includes a pair of guide rails 180 in the form of upstanding walls that extend upwardly from the upper planar face 154. The guide rails 180 are spaced opposite one another and are partially formed on opposite sides of the central bore 147 and are formed parallel to one another. Inner surfaces of the guide rails 180 define the upper regions of the U-shaped channel 170 and partially define the central bore 147. Each guide rails 180 includes a flange 190 that extends outwardly therefrom towards an outer circumferential edge of the body 142, with the flange 190 being parallel to the upper face 154 since the guide rail 180 is perpendicular to both the upper face 154 and the flange 190. The flange 190 does not extend completely to the outer circumferential edge but instead terminates prior thereto above the upper face 154 so as to define a guide slot or guide channel 200 between an underside of the flange 190 and the upper face 154 of the body 142. Since the body 142 has a circular shape, the ends of the flange 190 are not uniform, square ends but instead, the ends of the flange 190 are arcuate in shape and include a chamfered edge 192.

The body 142 of the second part 140 includes a pair of guide tangs 210. One guide tang 210 is formed on one respective upper planar surface of the flange 190 and extends upwardly therefrom. The guide tang 210 is an upstanding structure that extends upwardly from the upper surface of the flange 190. Inner surfaces of the guide tangs 210 are flush with the inner surfaces of the guide rails 180 so as to permit the rod 30 to be guided into the channel 170. It will be appreciated that the length of the guide tang 210 is less than the length of the guide rail 180. The guide tang 210 is preferably centered about the central bore 147 with the distances from one end of the guide tang 210 to one respective end of the guide rail 180 being the same. Similar to the guide rails 180 and more specifically, the flanges 190 thereof, the guide tangs 210 contain chamfered edges 212 at their ends.

On an inner surface(s) of the guide tang 210, an interference means 211 is formed on the inner surface to provide initial interference with the rod 30 as it is received within and between the tangs 210 and the guide rails 180. This resistance to the rod 30 caused by interference means 211 provides several functions, namely, (1) it offers a tactile and auditory snap or click feedback to the user indicating that the rod 30 has passed the tangs 210 and is in its proper position; (2) it transmits lateral load to the guidance tangs 210 to force them apart to permit passage of the rod 30, yet is not enough to deform them so the tangs 210 can rebound to their original disposition as spring-form elements; and (3) it provides enough resistance to confine and prevent the rod 30 from becoming accidentally upwardly dislodges from 140 before the cap 230 can be applied and locked into place.

For example, the interference means 211 is in the form of a protrusion that is formed on the inner surface of at least one of the guide tangs 210. The interference means 211 can be a vertical rail or a bump or other shaped protrusion. By placing the interference means 211 on the inner surface of one tang 210, the distance between the tangs 210 is reduced in the localized area where the interference member 211 is located and therefore, the rod 30 requires a degree of force to overcome the spring action of the tangs 210 before it can finally drop cleanly into the recessed channel 170 formed in the body 142 of the second part 140. Recessed channel 170 is slightly larger that the diameter of rod 30 so that the rod 30 can still be further positioned by freely sliding yet the rod 30 is not allowed to upwardly disconnect from the channel 170 freely. An alternative method of forming interference means 211 is to build tangs 210 in with a non-parallel receiving space that is smaller than the diameter of rod 30 at the top, or by bending the tangs 210 inwardly toward each other to form the interference means 211.

The anchor assembly 100 can also include the shim 220 that is built into the system during the manufacture of the system and in particular, the shim 220 is integrally coupled to the second part 140. The shim 220 is located between when the first part 130 and a portion of the second part 140. The shim 220 has a hollow section and includes an arcuate channel 222 (e.g., semicircular shape) that is formed along a top surface 224 of the shim 220. An inner space 226 formed in the shim 220 is defined by an inner, annular shaped, arcuate edge 227 that is configured to mate with rounded section 118 of the fastener head 112.

The shim 220 has a locating feature 229 that is designed to facilitate and locate the shim 220 relative to the second part 140. For example, the locating feature 229 can be in the form of a protrusion that extends outwardly and upwardly from the top surface 224 of the shim 220. The second part 140 preferably includes a complementary locating feature 141 in the form of an opening or like for receiving the locating feature 229 of the shim 220 so as to properly couple the shim 220 within the second part 140, whereby a complete channel is formed for receiving the rod 30 and a floor is formed for cradling and limiting the reception of the rod 30 within the second part 140. The opening 141 can extend completely through the second part 140 and be open along the upper planar surface 154. When the shim 220 mates with the second part 140, these two parts define the arcuate channel that hold and contain the rod 30. The locating features 141, 229 can be located outside of the guide channels 180 so as not to interfere with the reception of the rod 30 in the second part 140 or the opening 141 can open into and be in communication with one guide channel 180.

The shim 220 can thus be inserted after the fastener 110 is received within the first part 130 of the base 120. Since the underside of the shim 220 contains the hollow inner cavity 226 that is shaped to complement the rounded section 118 of the head 112, the shim 220 can further stabilize and locate the fastener 110 within the base 120.

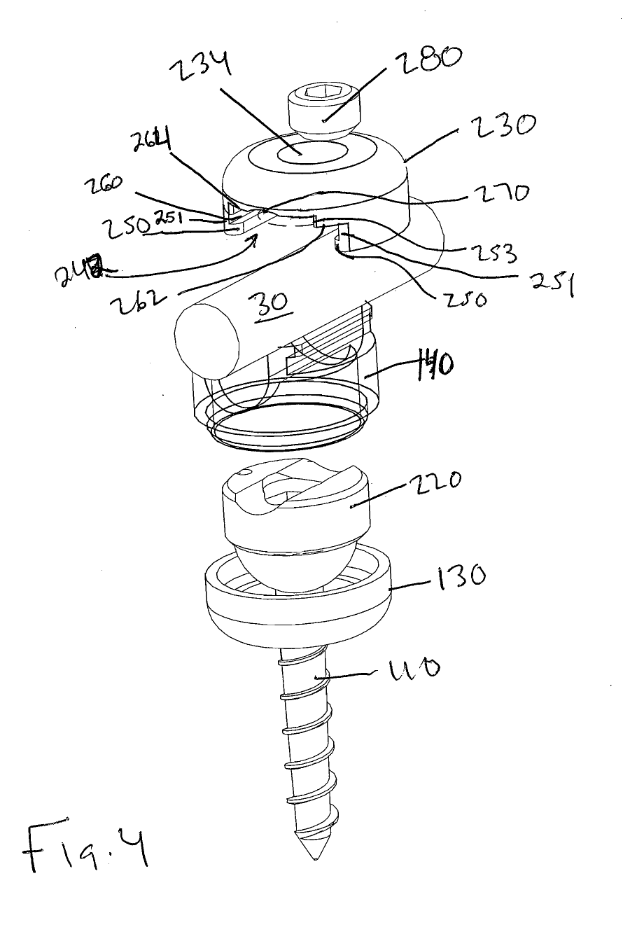

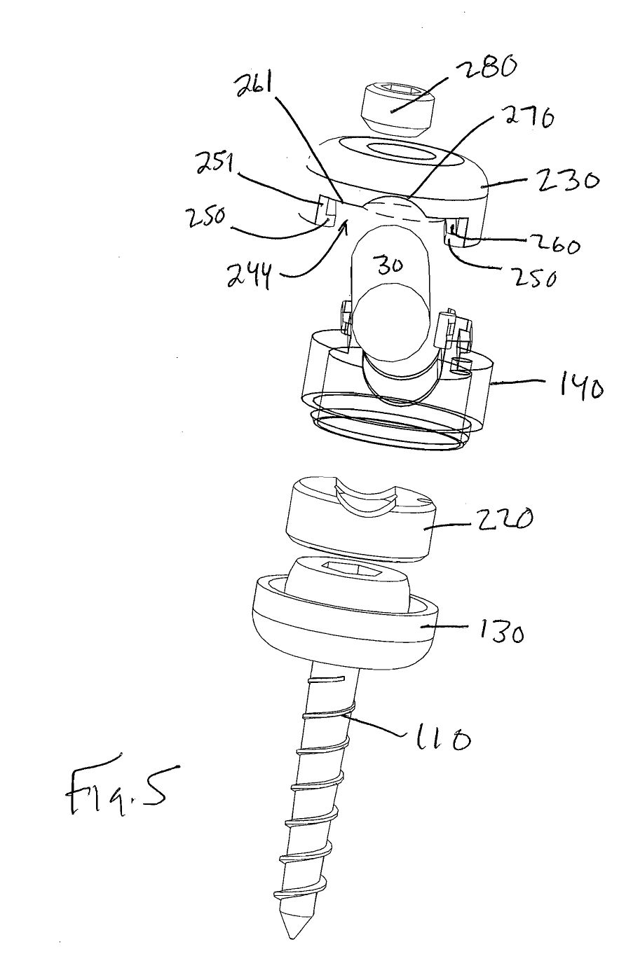

The anchor assembly 100 also includes a cap 230 that is configured to mate with the base 120 and more specifically, to the second part 140. The cap 230 is defined by a generally hollow body 232 that has a circular shape and has a central bore 234 that extends through the body 232 and through an upper face 233 of the body 232. The illustrated central bore 234 has a circular shape. The cap 230 includes an annular side wall 240 that is not uniform around the circumference of the cap 230. The side wall 240 has a first cut out 242 (FIG. 5) that defines a pair of interlocking rail structures 250 and a second cut out 244 (FIG. 4) that also defines the pair of interlocking rail structures 250. The second cut out 244 is located approximately opposite (180 degrees) the first cut out 242.

Each of the first and second cut outs 242, 244 includes a notch that defines the pair of interlocking rail structures 250. The interlocking rail structures 250 extend inwardly toward the hollow center of the cap 230 and in the illustrated embodiment have a square or rectangular cross-section. Preferably, the ends 249 of the rail structures 250 are chamfered ends 249 to facilitate the mating between the cap 230 and the second part 140. The upper and lower faces of the rail structure 250 are planar surfaces that are parallel to the upper face 233 of the body 232. The height of the rail structure 250 is complementary to the distance between the guide rail 180 and the upper surface 154 of the second part 140 since the rail structure 250 is intended to be slidingly received within this space between the guide rail 180 and the upper surface of the second part 140.

The first cut out 242 (FIG. 5) includes a space or slot 260 is formed between the upper surface of the rail structure 250 and a first horizontal wall 262 that is formed parallel to the upper surface of the rail structure 250. A first vertical wall or edge 251 is formed between the rail structure 250 and the first horizontal wall 262. This space 260 is configured to receive the flange 190 when the cap 230 is slidingly mated with the second part 140 of the base 120. The side wall 240 has a stepped construction in that a second vertical wall or edge 253 extends between the first horizontal wall 262 and a second horizontal wall 264, with the vertical edges 251, 253 being formed perpendicular to the horizontal walls 262, 264.

Along the length of the second horizontal wall 264, an arcuate wall section 270 is formed. This arcuate wall section 270 is spaced above the rod 30 when the cap 230 is mated with the second part 140. In combination with the U-shaped channel 170, the arcuate wall section 270 defines a circular boundary that surrounds the rod 30 and is designed to encapsulate the rod 30.

The second cut out 244 (FIG. 4) is similar to the first cut out 242 and therefore like elements are numbered alike, including the guide rails 250 that extend across opposing sides of the cap 230 and are formed also as part of the first cut out 242 and therefore, extend from one side to another opposite side of the cap 230. While the second vertical edges 253 are vertical and are perpendicular to the horizontal walls 262, 264, the second cut out 244 does not include the second vertical edge 253 and the second horizontal wall 264 but instead it includes a horizontal wall 261 that includes the rod engaging edge 270 that is in the form of an arcuate edge (semi-circular edge) formed along the length of the horizontal wall 261. In contrast to the walls structure of the first cut out 242, the horizontal wall 261 extends between and from the vertical edge or wall 251. As will be described below, the arcuate edge 270 is a rod engaging edge since when the cap 230 engages the second part 140 and the cap 230 is secured to the second part 140, the edge 270 is placed into engagement with the rod 30 so as to securely hold the rod 30 in place between the floor of the U-shaped channel 170 and the edge 270.

The arcuate wall section 270 also acts as a stop means that limits the lateral movement of the cap 230 relative to the base 120 (the second part 130). In particular, the arcuate wall section 270 is formed so that when the cap 230 is slidingly engaged with the second part 140 as described below, the degree or range of lateral movement of the cap 230 relative to the second part 140 is limited by engagement of the arcuate wall section 270 with ends of the guide tangs 210. Since the first cut out 242 does not include the stop means in the form of the arcuate wall section 270, the interlocking structures of the second part 140 are first received in the first cut out 242 and then the cap 230 is slidingly moved along the second part 140 until the stop means 270 engages the ends of the guide tangs 210 at which time, the central bore 234 formed in the cap 230 is axially aligned with the central bore 132. The stop means 270 also restricts the manner in which the cap 230 mates with the second part 140 in that if the interlocking features of the second part 140 are received within the second cut out 244, the cap 230 has only a limited, insufficient degree of lateral movement along the second part 140 until the stop means 270 contacts the ends of the guide tangs 210 (which results in a substantial portion of the cap 230 overhanging the second part 140) and the bore 234, 132 are not aligned.

The central bore 234 formed in the body 232 is in the form of a threaded bore that is designed to receive rod locking means 280 that is constructed to engage and apply a force to the underlying rod 30 so as to hold the rod 30 securely in place. In one embodiment, the rod locking means 280 is in the form of compression lock screw that is threadingly mated with threads of the central bore 234. The lock screw 280 can therefore be easily advanced within the bore 234 towards and into contact with the rod 30 and once it engages the rod 30, the further manipulation (tightening) of the lock screw 280 results in the rod 30 being compressed towards and into contact with the floor of the U-shaped channel 170. To release the rod 30 and permit movement of the rod 30, especially in the lateral direction or in an up-and-down direction, the lock screw 280 is simply retracted or “backed-off” from the cap 230.

To assemble the anchor assembly 100 to the target location, e.g., the pedicle, the base 120, and in particular, the first part 130 thereof, the fastener 110 is inserted through the central bore 132 so as to position the rounded shoulder 118 against the beveled section 134. The fastener 110 is then fastened to the bone (pedicle) using an appropriate tool, such as a driver. Next, the insert 220 is positioned over the head 112 so that the head 112 is received in a complementary cavity formed in the underside of the insert 220.

The second part 140 is attached to the first part 130 so that the insert 220 is located between the two parts 130, 140. The rod 30 is then received within the U-shaped channel 170. Since the guide tangs 210 are somewhat resilient in nature, the guide tangs 210 can flex outwardly as the rod 30 is received therebetween. As previously mentioned, the interference member 211 creates a local area of decreased channel width and therefore, the rod 30 does not freely drop into the U-shaped channel 170 but instead, some force needs to be applied to the rod 30 against the interference member 211 to cause the interference member 211 to flex outwardly and thereby permit free passage of the rod 30 into the U-shaped channel 170. Once the rod 30 clears the interference member 211, the guide tangs 210 flex back to their original position and thereby effectively capture the rod 30 in the U-shaped channel 170 since the rod 30 cannot freely become dislodged from the U-shaped channel 170 since the interference member 211 presents an obstruction.

The cap 230 is then attached to the base 110 by slidingly yet removably mating the cap 230 with the second part 140. This is accomplished by inserting the rails structures 250 within the guide slots 200 between the flange 190 and upper face 154 of the body 142. The ends of the rail structures 250 that are formed at the first cut out 242 are inserted first into the guide slots 200. Once the rail structures 250 are inserted into the guide slots 200, the cap 230 can slidingly travel across the upper face 154 of the body 142. It will be appreciated that the flanges 190 are disposed above the rail structures 250 and limit the vertical (up-and-down) movement of the cap 230 since the rail structures 250 are captured within the guide slots 200. The chamfered edges 212 of the guide tangs 210 and the chamfered ends 249 of the rail structures 250 help facilitate the reception of the cap 230 into the second part 140 of the base 120.

When the top planar surface of the rail structures 250 seat against the underside planar surface of the flange 190, there is a slight gap 271 formed between the underside or bottom planar surface of the rail structures 250 and the upper face 154 of the body 140 of the second part 140. This gap 271 permits the cap 230 to freely and slidingly travel along the upper face 154 of the body 140 before the lock screw 280 is tightened within the threaded bore 234 of the body 232 and into engagement with the rod 30. Thus, after the lock screw 280 is tightened into engagement with the rod 30, if it is desired to laterally move the cap 230 relative to the base 120, then the lock screw 280 is backed off (retracted) from the rod 30 and the cap 230 can slide across the planar upper face 154 in part due to the presence of the gap 271.

As the rail structures 250 slide within the guide slots 200, the guide tangs 210 are disposed between the side wall 240 of the cap 230. In particular, the guide tangs 210 are disposed between the second vertical edges 253. The cap 230 is laterally moved along the planar upper face 154 until the stop means 270 makes contact with the ends of the guide tangs 210 as previously mentioned at which time further lateral movement of the cap 230 relative to the base 120 is prevented. Once the stop means 270 engages the ends of the guide tangs 210, the bores 134, 234 are axially aligned with one another and since the rail structures 250 are contained in the guide slots 200, the cap 230 is prevented from moving excessively in an up-and-down manner.

Once the bores 134, 234 are axially aligned, the lock screw 280 can be inserted and threadingly secured within the bore 234. As the lock screw 280 is received in the threaded bore 234 and tightened using a driver or the like, the lock screw 280 continuously moves closer to and finally into engagement with the rod 30 that is being cradled within the U-shaped channel 170. The lock screw 280 serves to lock and secure the rod 30 in place with respect to the base 110 which is itself fixed in place at the target location as by being attached to the pedicle using the fastener 110.

It will be appreciated that any number of other stop means of a different construction than the stop means 270 can be used so long as the stop means serves to limit the degree of lateral travel of the cap 230 relative to the base 110. For example, the base 110 itself (e.g., the second part 140) can include a protrusion or the like along a peripheral edge proximate to or along the guide slot 200 such that the rail structure 250 contacts the protrusion when the bores 134, 234 are aligned and the cap 230 is in a proper position relative to the base 110.

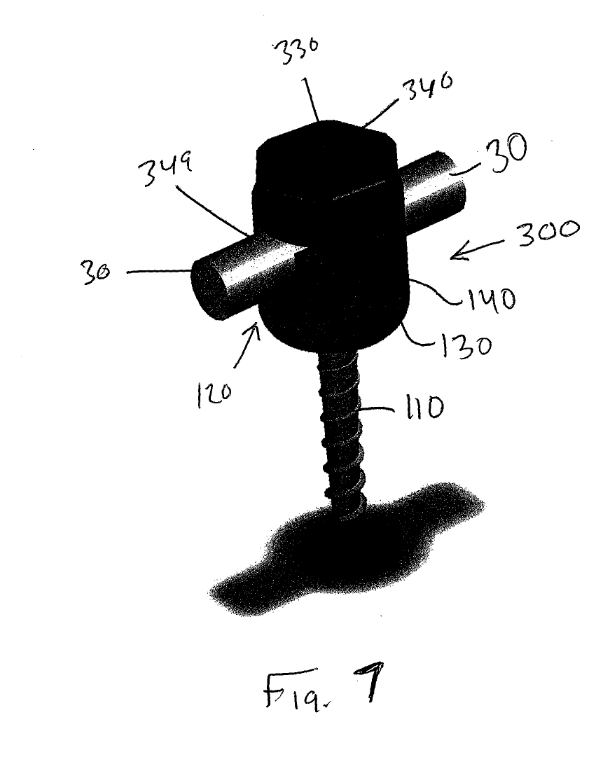

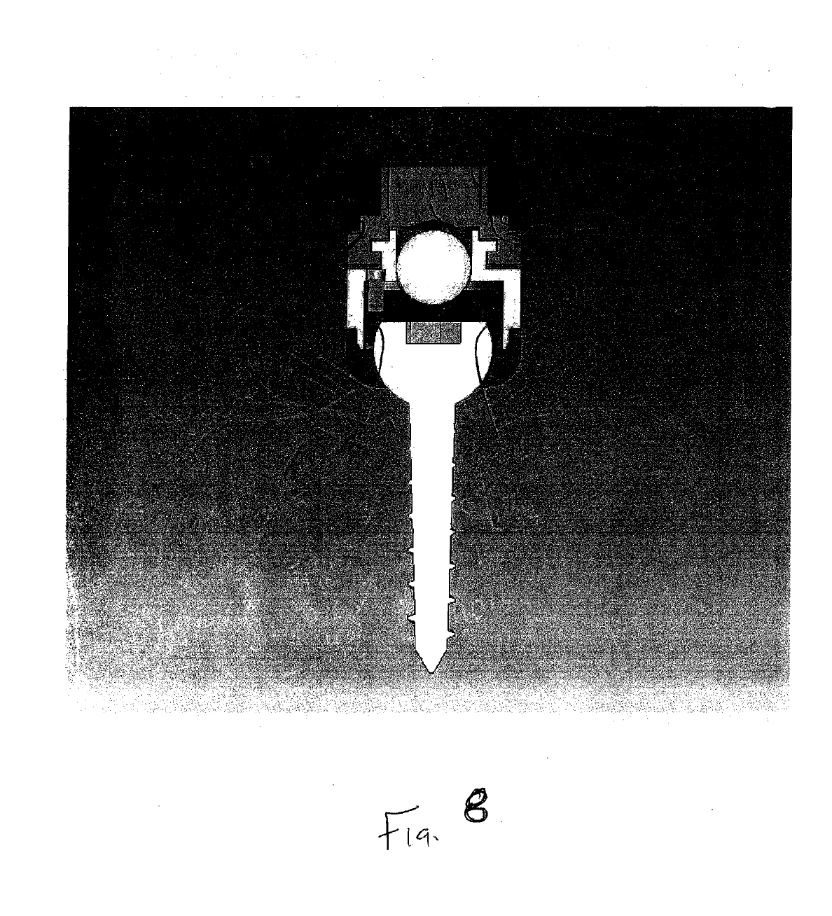

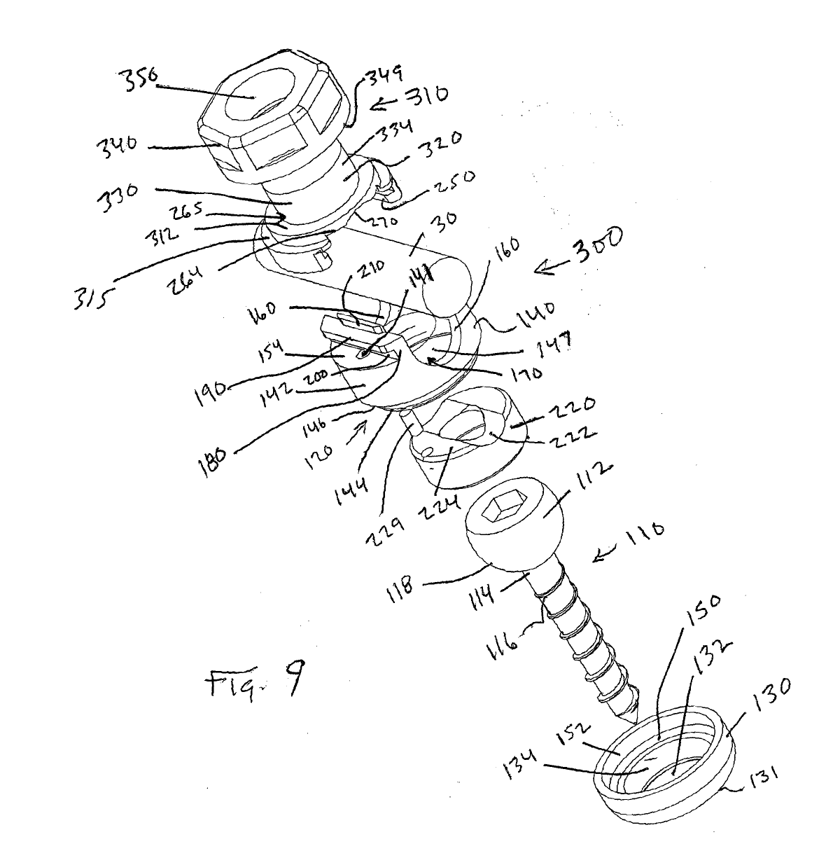

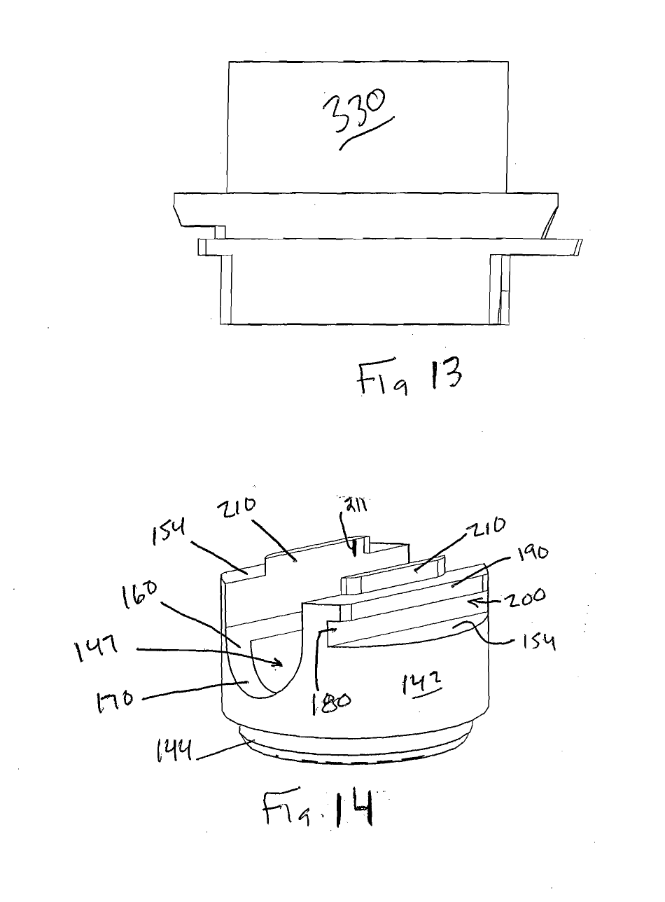

FIGS. 7-14 illustrate an anchor assembly 300 according to another embodiment. The anchor assembly 300 has a number of features and parts that are either identical to or similar to those discussed in relation to the anchor assembly 100 and therefore, like elements are numbered alike. The anchor assembly 300 includes the fastener 110, the base 120 including the first and second parts 130, 140, and the insert 220, as well as the rod 30. The difference between the anchor assembly 300 and the anchor assembly 100 is the design of the cap. More specifically, the anchor assembly 300 includes a cap 310 that is slidingly mated with the base 120 similar to how the cap 230 slidingly mates with the base 120.

The cap 310 is actually formed of two parts, namely, a cap base 320 that includes a threaded post 330 and a threaded cap 340. The cap base 320 is very similar to the cap 230 in that it includes interlocking, coupling features for mating with the second part 140 of the base 120. In fact, the cap base 320 includes the rail structures 250 and can also include the stop means 270. The main difference between the cap 310 and the cap 230 is that the cap 310 does not includes the threaded bore 234 since the means for engaging the rod 30 in the cap 310 are different than the means used in the cap 230. In particular, the cap 310 does not use the lock screw 280 since it does not include a threaded bore formed therethrough.

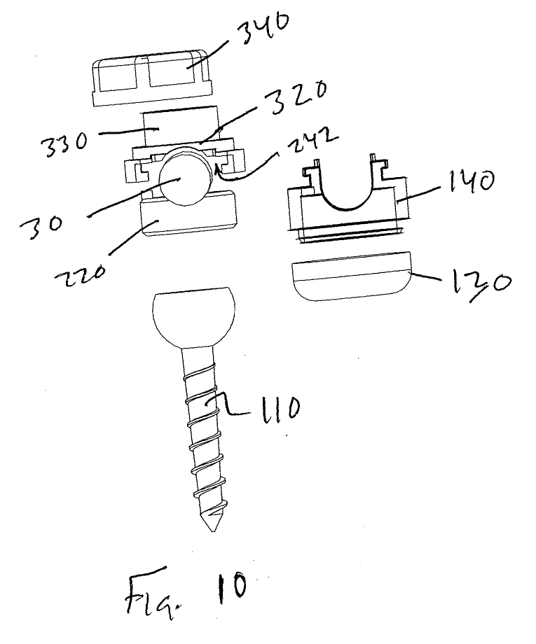

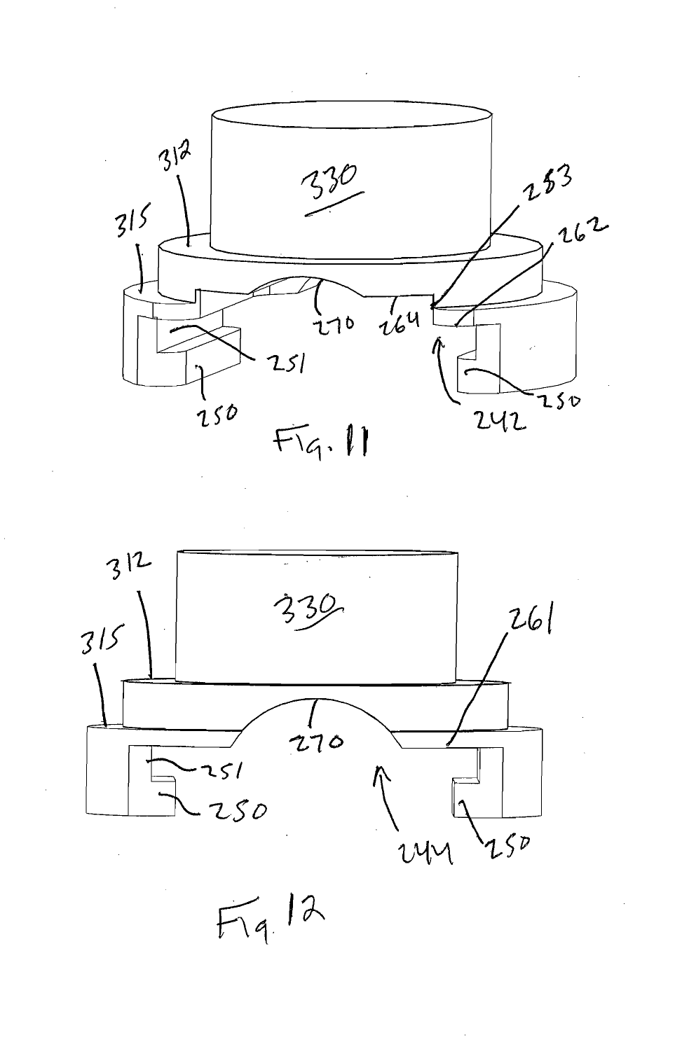

As with the first embodiment of FIGS. 2-5, the cap base 320 includes the first cut out 242 (FIG. 11) and the second cut out 244 (FIG. 12) and the different associated wall structures. The second horizontal wall 264 of the cap base 320 that is formed as part of the cut outs 242 includes the arcuate edge 270 that has a complementary shape (e.g., semicircular) as the rod 30 and the arcuate edge 270 is also formed in the horizontal edge or wall 261 of the second cut out 244. An upper surface 312 of the second horizontal wall 264 defines a planar platform from which the threaded post 330 is coupled to and extends outwardly therefrom. The threaded post 330 can be integrally formed with the cap base 320 as a single part or it can be coupled thereto, as by welding or the like. The threaded post 330 is in the form of a threaded boss or protrusion that has threads formed along an outer surface 334 thereof. In the illustrated embodiment, the threaded post 330 has a circular shape; however, this is merely one exemplary shape and the post 330 can be formed in any number of other shapes so long as the threaded cap 340 has a complementary shape. The post 330 is preferably centrally located on the platform 312.

The threaded cap 340 has a substantially hollow body 342 that includes a threaded bore 350 formed therethrough, preferably, through the center thereof. An underside of the body 342 has an annular shoulder 344 formed thereat and is defined by an inner surface 346 of a bottom section 343 of the body 342 and an annular surface 348 that is formed around the threaded bore 350 and formed at a right angle with the inner surface 346 that is radially outward from the threaded bore 350 and from the annular surface 348. It will be appreciated that an inner diameter of the bottom section 343 is greater than a diameter of the threaded bore 350 of the threaded cap 340.

The annular shoulder 344 is designed to mate with a shoulder 265 defined in part by the second horizontal wall 264 so that the annular surface 348 seats against the upper surface 312 of the second horizontal wall 264. The bottom section 343 circumferentially surrounds the platform 312 such that the second horizontal wall 264 is disposed between the bottom section 343 of the threaded cap 340. Both the platform 312 and a second annular platform 315 that is defined as an upper surface of the first vertical wall 251 and is formed circumferentially around the second horizontal wall 264 limit the degree of travel of the threaded cap 340 on the threaded post 330. In other words, the threaded cap 340 can only be tightened on the threaded post 330 a certain degree until the cap 340 will engage (‘bottom out”) and seat against the cap base 320.

The threaded cap 340 includes a bottom edge 349 that serves as the means for impinging and applying a retaining force to the rod 30 that underlies the cap 310. More specifically, the bottom edge 349 engages the rod 30 in two opposing places, namely at the two ends of the U-shaped channel 170 where the rod 30 is exposed, and applied a force against the rod 30 that is captured in the U-shaped channel 170.

The rod 30 is dimensioned so that when it seats within the U-shaped channel 170, a top portion of the rod 30 is disposed above the second annular platform 315 so that as the threaded cap 340 is tightened on the threaded post 330 and travels downward toward the rod 30 and the base 120, the bottom edge 349 of the threaded cap 340 contacts the top portion of the rod 30 in the two locations that are 180 degrees apart from one another. Thus, as the threaded cap 340 is screwed onto the threaded post 330, the bottom edge 349 comes into contact with the rod 30 as shown in FIGS. 7 and 8 resulting in the cap 340 applying a force to the rod 30 that is sufficient to result in the rod 30 being securely held within the U-shaped channel 170 and be securely attached to the anchor assembly 300. It will also be appreciated that, as shown in FIG. 8, a slight gap or space 351 is formed between the bottom edge 349 and the second annular platform 315 when the threaded cap 340 is in the fully engaged position relative to the post 330 and is in full engagement with the rod 30. The engagement of the threaded cap 340 with the rod 30 provides a means for securely retaining the rod 30 within the U-shaped channel 170 so that lateral movement of the rod 30 is prevented. To laterally readjust the rod 30 within the implant 300, the threaded cap 340 is simply backed off or retracted on the post 330 until the bottom edge 349 is no longer applying an impinging force on the rod 30. Once properly readjusted, the threaded cap 340 is again tightened on the post 330 until the bottom edge 349 comes into contact with the rod 30.

It will be appreciated that the anchor assembly 100, 300 according to the present invention offer an improved design relative to the prior art designs and more particularly, the anchor assembly 100, 300 are less complex and require less tools in order to securely attach the anchor assembly 100, 300 to the target location, such as the pedicle, and also offers an improved, less complex means for locking the rod 30 relative to the anchor assembly 100, 300. The sliding action of the respective caps relative to the base portions permits the cap to be easily and quickly coupled to the base and the stop means associated with the cap and base portion controls and limits the degree of lateral movement of the cap relative to the base so as to permit the cap to be easily moved into a position where the through bores of the cap and base are axially aligned so as permit the desired retention of the rod in the base.

It will also be appreciated that a crosslink, such as the one disclosed in the '555 patent, can be used to stabilize the rod members 30 against torsional rotational rotation. It is preferable that two crosslinks be used so as to form a rectangular construct. Each crosslink includes two clamps each secured to the main rods. Each clamp includes a rod receiving channel which accommodates the rod and is locked into position relative thereto in the manner described in detail in the '555 patent.

One exemplary method of therapy for use of one of the anchor assemblies of the present invention is described below. A longitudinal posterior midline incision is made over the spine. The incision is carried through the subcutaneous tissue and the fascia to the tips of the spinous processes. Subperiosteal dissection is performed over the laminae and transverse processes. The facet capsule and articular cartilage are removed in preparation for fusion. The pedicle is located using standard tools, such as an awl and a pedicle hole is made using a pedicle probe or the like. The pedicle probe is inserted into the hold initially created by the awl and is manipulated by the surgeon until he/she feels a relatively soft gritty sensation of the cancellous bone within the pedicle and vertebral body during this procedure. The depth of the hole is determined by using graduated markings on the pedicle probe and then the appropriate size screw is then chosen for that particular pedicle.

After the hole has been created, the size of the anchor assembly is selected depending on the height needed for the rod to rest above the fusion bed. The surgeon then sequentially inserts an appropriate transpedicular screw 110 and the first and second parts 130, 140 of the base 120 are assembled, with the insert 220 being disposed therebetween. An appropriate length of rod 30 is chosen and countered to fit within the anchor assemblies and is then placed therein using an appropriate tool. The respective cap is then slidingly received and mated to the base such that the cap is properly positioned on the base and then depending upon the type of anchor assembly being used, either the set screw or the threaded cap is tightened using an appropriate tool resulting in a force being applied to the rod. The procedure is repeated on the other side of the spine over the same number of vertebral levels.

As previously mentioned, the crosslinks can be applied for added torsional stability. The crosslink can be formed of two clamps, each of which is secured to one of the two main rods with set screws. The clamps are then bridged together by the crosslink (e.g., a Steinmann pin) which is cut to length equivalent to the distance between the clamps. The crosslink is thus secured to the clamps using sets screws. It is recommended that at least two sets of crosslinks are used to provide a more stable construct.

It will be appreciated by persons skilled in the art that the present invention is not limited to the embodiments described thus far with reference to the accompanying drawings; rather the present invention is limited only by the following claims.

What is claimed is:

1. An anchor assembly for use with a stabilizer system for stabilizing one or more spinal bone comprising:
a fastener for securing the anchor assembly to the one or more spinal bone segments;
an anchor base having a first coupling feature including guide slots and a recessed channel for receiving the stabilizer and a first bore and a first cavity including a seat for receiving a head of the fastener;
a cap having a bore formed therethrough, a second coupling feature including rail structures that are laterally received in the guide slots, and a stop element that acts with the first coupling feature to limit the lateral movement of the cap in the guide slots and causes the first bore and the threaded bore to be aligned in a first position; and
a lock member that is received within the bore of the cap and can be engaged with the stabilizer to lock and retain the stabilizer in place within the anchor assembly.

2. The anchor assembly of claim 1, wherein the fastener extends through the first bore and when the head engages the seat, the fastener extends beyond the anchor base, the cap bore comprising a threaded bore and the lock member is threadingly received in the threaded cap bore.

3. The anchor assembly of claim 1, wherein the stabilizer comprises an elongated metal rod.

4. The anchor assembly of claim 1, wherein the anchor base comprises a first part and a second part that is coupled to the first part with the head of the fastener being disposed therebetween, the first part having the first bore formed therethrough and the seat is in the form of an annular beveled surface complementary to a rounded surface of the head of the fastener, the first coupling feature including a pair of outwardly extending flanges that in combination with a planar upper face of a body of the second part define the guide slots, the rail structures of the cap being defined by a pair of inwardly extending flanges that are received in the guide slots.

5. The anchor assembly of claim 4, wherein the outwardly extending flanges, the inwardly extending flanges and the planar upper face are all parallel to one another with a height of each guide slot being greater than a height of each rail structure so that the cap has some degree of vertical movement with respect to the base.

6. The anchor assembly of claim 4, wherein a side wall of the body of the second part includes the recessed channel that is formed between the pair of outwardly extending flanges and extends below the planar upper face of the body of the second part.

7. The anchor assembly of claim 6, wherein the recessed channel is defined by a pair of U-shaped notches formed in the side wall adjacent the first bore for cradling the elongated stabilizer, wherein a maximum width of the notch is equal to or greater than a diameter of the elongated stabilizer.

8. The anchor assembly of claim 4, wherein the second part includes a pair of upstanding guide tangs that extend outwardly from an upper surface of the pair of the outwardly extending flanges, the guide tangs being formed on opposite sides of the first bore.

9. The anchor assembly of claim 8, wherein at least one guide tang includes an interference means that is formed on an inner surface thereof that faces the first bore, wherein a distance between the interference member and the opposing guide tang is less than a diameter of the elongated stabilizer so as to prevent the stabilizer from freely leaving the recessed channel.

10. The anchor assembly of claim 9, wherein the interference member comprises a protrusion formed on the inner surface of the guide tang.

11. The anchor assembly of claim 9, wherein the stop element comprises a portion of the cap that contacts ends of the guide tangs to limit the lateral movement of the cap in the guide slots in the first position resulting in the first bore and the threaded bore being aligned.

12. The anchor assembly of claim 4, further including an insert that is disposed between the first and second parts of the base, the insert having an opening formed therethrough that defines a section of the first bore and an upper surface of the insert includes an arcuate channel formed therein that intersects the opening and is configured to serve as a bottom seat for receiving and supporting the elongated stabilizer.

13. The anchor assembly of claim 11, wherein the insert has a cavity formed in an underside thereof that includes an annular beveled surface that is complementary to and engages a surface of the head of the fastener.

14. The anchor assembly of claim 11, wherein the upper surface of the insert includes a first locating guide feature that mates with a second locating guide feature that is part of the second part of the base so that the insert can only be received within an underside cavity formed in the second part when the first and second locating guide features are in registration.

15. The anchor assembly of claim 14, wherein the first locating guide feature comprises a post that extends outwardly from the insert and the second locating guide feature comprises a through hole formed through the second part.

16. The anchor assembly of claim 15, wherein the through hole is in communication with and opens into one guide slot.

17. The anchor assembly of claim 1, wherein the rail structures are in the form of inwardly extending flanges that are slidingly received in the guide slots, the guide slot having a ceiling that is defined by an outwardly extending flange such that vertical movement of the inwardly extending flange in the guide slot is restricted by the outwardly extending flange, the second coupling feature further including a slot for receiving the outwardly extending flange such that in the first position, the outwardly extending flange overlies the inwardly extending flange in a parallel manner.

18. The anchor assembly of claim 17, wherein in the first position, the inwardly extending flange seats against an underside of the outwardly extending flange such that a gap is formed between a bottom edge of the cap and the base.

19. The anchor assembly of claim 1, wherein the rod locking member comprises a lock screw that applied a force perpendicular to the elongated stabilizer to lock it in place within the base by threadingly engaging the bore of the cap.

20. The anchor assembly of claim 1, wherein there is an annular gap formed between a bottom edge of the cap and the base in the first position when the rod locking member applies a force to the elongated stabilizer resulting in the elongated stabilizer being securely fixed in place.

21. The anchor assembly of claim 8, wherein the distance between a portion of the guide tangs is slightly less than the diameter of the elongated stabilizer so as to prevent the stabilizer from freely leaving the recessed channel.

22. The anchor assembly of claim 21, wherein the guide tangs comprise a spring form rod receiving means.

23. An anchor assembly for use with a stabilizer system for stabilizing one or more spinal bone segments comprising:
a fastener for securing the anchor assembly to the one or more spinal bone segments;
an anchor base that includes a seat for receiving a head of the fastener and a first coupling feature including guide slots and shaped slots for receiving the elongated stabilizer, the shaped slots defining an axis that intersects the first bore;
a cap including a base having a post and a second coupling feature including structures that permit the cap to be laterally received and moved within the guide slots while restricting vertical movement of the cap; and
a cap fastener coupled to the post and can be driven along the post into engagement with the stabilizer to lock and retain the elongated stabilizer in place within the anchor assembly.

24. The anchor assembly of claim 23, wherein the anchor base include a first bore and first cavity that includes the seat such that when the fastener head engages the seat, the fastener extends beyond the anchor base.

25. The anchor system of claim 23, wherein the post of the cap base comprises a threaded post with threads on an exterior surface thereof, the cap fastener threadingly mating with the threaded post.

26. The anchor assembly of claim 23, wherein the cap base includes a first platform and a second platform that is formed circumferentially around the first platform, the cap fastener having a first annular surface for seating against the first platform when the cap fastener is in a fully engaged position and a bottom flange member that is disposed above but spaced from the second platform in the fully engaged position.

27. The anchor assembly of claim 25, wherein the cap fastener has a central threaded bore that threading mates with external threads formed on the threaded post, the cap fastener contacting and applying a force to the elongated stabilizer at two opposite locations where the shaped slots are formed.

28. The anchor assembly of claim 27, wherein the force is a force perpendicular to the elongated stabilizer.

29. The anchor assembly of claim 23, wherein the stabilizer comprises an elongated metal rod.

30. The anchor assembly of claim 23, wherein the anchor base comprises a first part and a second part that is coupled to the first part with the head of the fastener being disposed therebetween, the first part having the first bore formed therethrough and the seat is in the form of an annular beveled surface complementary to a rounded surface of the head of the fastener, the first coupling feature including a pair of outwardly extending flanges that in combination with a planar upper face of a body of the second part define the guide slots, the structures of the second coupling feature comprising elongated rails defined by a pair of inwardly extending flanges that are received in the guide slots.

31. The anchor assembly of claim 30, wherein the outwardly extending flanges, the inwardly extending flanges and the planar upper face are all parallel to one another with a height of each guide slot being greater than a height of each rail structure so that the cap base has some degree of vertical movement with respect to the anchor base.

32. The anchor assembly of claim 30, wherein a side wall of the body of the second part includes the shaped slots that are formed between the pair of outwardly extending flanges and extends below the planar upper face of the body of the second part.

33. The anchor assembly of claim 32, wherein the shaped slots are defined by a pair of U-shaped notches formed in the side wall adjacent a first bore formed through the anchor base, wherein a maximum width of the notch is equal to or greater than a diameter of the elongated stabilizer.

34. The anchor assembly of claim 30, wherein the second part includes a pair of upstanding guide tangs that extend outwardly from an upper surface of the pair of the outwardly extending flanges, the guide tangs being formed on opposite sides of a first bore formed through the anchor base.

35. The anchor assembly of claim 34, wherein at least one guide tang includes an interference member that is formed on an inner surface thereof that faces the first bore, wherein a distance between the interference member and the opposing guide tang is less than a diameter of the elongated stabilizer so as to prevent the stabilizer from freely entering the recessed channel.

36. The anchor assembly of claim 35, wherein the interference member comprises a protrusion formed on the inner surface of the guide tang.

37. The anchor assembly of claim 34, further including a stop element defined by a portion of the cap base that contacts ends of the guide tangs to limit the lateral movement of the cap base in the guide slots in the first position resulting in the first bore and the threaded bore being aligned.

38. The anchor assembly of claim 30, further including an insert that is disposed between the first and second parts of the anchor base, the insert having an opening formed therethrough that defines a section of the first bore and an upper surface of the insert includes an arcuate channel formed therein that intersects the opening and is configured to serve as a bottom seat for receiving and supporting the elongated stabilizer.

39. The anchor assembly of claim 38, wherein the insert has a cavity formed in an underside thereof that includes an annular beveled surface that is complementary to and engages a surface of the head of the fastener.

40. The anchor assembly of claim 38, wherein the upper surface of the insert includes a first locating guide feature that mates with a second locating guide feature that is part of the second part of the base so that the insert can only be received within an underside cavity formed in the second part when the first and second locating guide features are in registration.

41. The anchor assembly of claim 40, wherein the first locating guide feature comprises a post that extends outwardly from the insert and the second locating guide feature comprises a through hole formed through the second part.

42. The anchor assembly of claim 41, wherein the through hole is in communication with and opens into one guide slot.

43. The anchor assembly of claim 23, wherein the rail structures are in the form of inwardly extending flanges that are slidingly received in the guide slots, the guide slot having a ceiling that is defined by an outwardly extending flange such that vertical movement of the inwardly extending flange in the guide slot is restricted by the outwardly extending flange, the second coupling feature further including a slot for receiving the outwardly extending flange such that in the first position, the outwardly extending flange overlies the inwardly extending flange in a parallel manner.

44. The anchor assembly of claim 43, wherein in the first position, the inwardly extending flange seats against an underside of the outwardly extending flange such that a gap is formed between a bottom edge of the cap fastener and the cap base.

45. The anchor assembly of claim 23, wherein there is an annular gap formed between a bottom edge of the cap base and the anchor base in the first position when the retaining element applies a force to the elongated stabilizer resulting in the elongated stabilizer being securely fixed in place.

46. A fixation device for the stabilization of one or more bone segments comprising:
at least two anchor assemblies according to claim 1 and an elongated stabilizing rod that is captured and retained within the at least two anchor assemblies resulting in stabilization of the one or more bone segments.
